(19) United States
(12) Patent Application Publication
(10) Pub. No.: US 2014/0275756 A1
(43) Pub. Date: Sep. 18, 2014

(54) Devices, Systems and Methods for Treating Benign Prostatic Hyperplasia and Other Conditions

(71) Applicant: Neotract, Inc., Pleasanton, CA (US)

(72) Inventors: Theodore C Bender, Oakland, CA (US); Ling-Kang Tong, Fremont, CA (US); Joseph Catanese, III, San Leandro, CA (US); Floria Cheng, San Francisco, CA (US); Jolene Cutts, San Francisco, CA (US); James Freitas, San Ramon, CA (US); Robert George, San Jose, CA (US); Theodore C. Lamson, Pleasanton, CA (US); Daniel Merrick, Dublin, CA (US); Earl A. Bright, II, Los Altos, CA (US); Arthur Ferdinand, San Jose, CA (US); Timothy A. Limon, Cupertino, CA (US); Brian Shiu, Sunnyvale, CA (US); Shuji Uemura, San Francisco, CA (US); Zachary Warder-Gabaldon, Palo Alto, CA (US); Michael Gearhart, Fremont, CA (US); Matthew McLean, San Francisco, CA (US); James Niederjohn, San Jose, CA (US); Brian Y. Tachibana, Oakland, CA (US); Ben Thompson, San Carlos, CA (US); Andrew L. Johnston, Redwood City, CA (US); John Stiggelbout, Sausalito, CA (US)

(73) Assignee: Neotract, Inc., Pleasanton, CA (US)

(21) Appl. No.: 14/209,633

(22) Filed: Mar. 13, 2014

Related U.S. Application Data
(60) Provisional application No. 61/800,303, filed on Mar. 15, 2013.

(51) Int. Cl. A61F 2/00

(52) U.S. Cl. CPC A61F 2/0045
USPC 600/37

(57) ABSTRACT

Devices, systems and methods for compressing, cutting, incising, reconfiguring, remodeling, attaching, repositioning, supporting, dislocating or altering the composition of tissues or anatomical structures to alter their positional or force relationship to other tissues or anatomical structures. In some applications, the invention may be used to improve patency or fluid flow through a body lumen or cavity (e.g., to limit constriction of the urethra by an enlarged prostate gland).

### CROSS-REFERENCE TO RELATED APPLICATIONS

This application claims priority to and the benefit of pending U.S. Provisional Application No. 61/800,303 filed Mar. 15, 2013 entitled “Devices, Systems And Methods For Treating Benign Prostatic Hyperplasia And Other Conditions,” which application is incorporated herein by reference in its entirety.

### BACKGROUND

Benign Prostatic Hyperplasia (BPH) is one of the most common medical conditions that affect men, especially elderly men. It has been reported that, in the United States more than half of all men have histopathologic evidence of BPH by age 60 and, by age 85, approximately 9 out of 10 men suffer from the condition. Moreover, the incidence and prevalence of BPH are expected to increase as the average age of the population in developed countries increases. Despite extensive efforts in both the medical device and pharmaco-therapeutic fields, current treatments remain only partially effective and are burdened with significant side effects. Thus, there remains a need for the development of new devices, systems and methods for treating BPH as well as other conditions in which one tissue or anatomical structure impinges upon or compresses another tissue or anatomical structure.

### SUMMARY

Embodiments disclosed herein include a system for treatment of a prostate. The system includes a first anchor configured to be placed on an outer surface of a capsule of the prostate. The first anchor is fixed to an end portion of a flexible connector. The system includes a second anchor configured to be placed on an outer surface of a capsule of the prostate. The second anchor slidably engages the flexible connector. The second anchor is configured to allow the connector to slide only in one direction. A portion of the connector is placed across a urethral surface of the prostate.

In some embodiments, the first anchor is configured to toggle about the connection point with the end portion of a flexible connector. In some embodiments, the second anchor is configured to create a 180 degree bend in the flexible connector. In some embodiments, the second anchor comprises a rounded projection and a stopping latch. In some embodiments, the system includes a wire having an angled distal end, and the angled distal end of the wire cooperates with the stopping latch of the second anchor to eject the second anchor from the system. In some embodiments, the end of the first anchor is angled. In some embodiments, the system includes a wire including an angled distal end, and the angled distal end of the wire cooperates with the angled end of the first anchor to eject the first anchor from the system. In some embodiments, multiple second anchors are loaded within the system. In some embodiments, the first anchor and the second anchor are carried by a penetrating member. In some embodiments, the first anchor is ejected from a portion of the penetrating member proximal to a distal end portion of the penetrating member. In some embodiments, the second anchor is ejected from a portion of the penetrating member proximal to a distal end portion of the penetrating member.

Embodiments disclosed herein include method of placing anchors proximate prostate tissue. The method includes placing a delivery system proximate a prostate gland. The delivery system includes a penetrating member, a first anchor, a second anchor, and a flexible connector. The first anchor is fixed to the connector and the second anchor is configured to allow the connector to slide only in one direction. The method includes advancing the penetrating member to penetrate the prostatic capsule at a first location and deploying the first anchor to a position proximate an outer surface of the prostatic capsule. The method includes advancing the penetrating member to penetrate the prostatic capsule at a second location and deploying the first anchor to a position proximate an outer surface of the prostatic capsule. The connector connects the first anchor and second anchor and is positioned such that a segment of the connector engages a portion of the prostatic urethra of the prostate gland.

In some embodiments, the system further includes a deployment wire and the first anchor is deployed by engaging an end of the deployment wire with an end of the first anchor. In some embodiments, the system further includes a deployment wire and the second anchor is deployed by engaging an end of the deployment wire with an end of the second anchor. In some embodiments, the method further includes setting a tension on the connector. In some embodiments, the tension is maintained by features on the second anchor. In some embodiments, the tension is maintained by the cooperation of a rounded projection and a stopping latch on the second anchor.

### BRIEF DESCRIPTION OF THE DRAWINGS

FIG. 1A illustrates an anchor according to an embodiment displacing and/or compressing tissue.

FIG. 1B illustrates an anchor according to an embodiment for providing an anchor point for a connector, such as suture.

FIG. 1C illustrates implantation of an anchor according to an embodiment.

FIG. 1D illustrates an anchor according to an embodiment for providing an anchor point for a connector, such as suture.

FIG. 1E and 1E′ illustrate an anchor according to an embodiment in which tissue is displaced towards bone.

FIG. 1F through 1F″″ illustrate placement of a one-piece anchor according to an embodiment.

FIG. 1G through 1G″ illustrate placement of a one-piece anchor according to an embodiment.

FIG. 2A, 2B and 2B′ illustrate embodiments of anchors configured to securely attached to a connector such as a suture.

FIG. 3A through 3C illustrate delivery of an anchor system that includes a connector such as a suture according to an embodiment.

FIG. 4A and 4B illustrate an alternate anchor system that includes a connector such as a suture according to an embodiment.

FIG. 5A through 5C illustrate a tissue-piercing anchor including a depth control feature according to an embodiment.

FIG. 6A and 6B illustrate a tissue-approximation system with a tensioning element according to an embodiment.

FIG. 7A and 7B illustrate a method for displacing prostatic tissue using suture.

FIG. 8A and 8B illustrate embodiments of delivery of an anchor that is detachable from a delivery system.

FIG. 9A and 9B illustrate embodiments of a one-piece anchor.

FIG. 9C, 9C′ and 9D illustrate embodiments of an anchor that is formed in-situ.

FIG. 10A through 10C illustrate embodiments of staple-like anchors and placement of such anchors in tissue.

FIG. 11A and 11B illustrate embodiments of anchors that include barb-like features.

FIG. 12A through 12C illustrate embodiments of staple-like anchors and placement of such anchors in tissue.

FIG. 12D illustrates a system and method for forming staple-like anchors in situ according to an embodiment.

FIG. 13A through 13E illustrate systems for deploying a series of anchors according to various embodiments.

FIG. 14A and 14B illustrate multiple anchor loading systems according to various embodiments.

FIG. 15A and 15B illustrate multiple anchor delivery systems according to various embodiments.

FIG. 16 through 18 illustrate various embodiments of methods to retract, displace, or compress prostatic tissue using configurations of anchors and suture.

FIG. 19 through 21B illustrate various embodiments of systems and methods to tighten and/or secure suture in configurations of anchors and suture.

FIG. 22A through 22D illustrate various embodiments of methods to retract, displace, or compress prostatic tissue using configurations of multiple anchors and suture.

FIG. 23 illustrates a delivery system for placing an anchor according to an embodiment.

FIG. 24A through 24C illustrate cross-sectional views of various embodiments of a delivery system for placing an anchor.

FIG. 25A and 25B illustrate a device for holding a delivery device in position at a tissue location according to an embodiment.

FIG. 26 illustrates various embodiments of methods to secure an anchor to tissue.

FIG. 27 illustrates a delivery needle for delivering anchors according to an embodiment.

FIG. 28 through 29B illustrate suture anchors according to an embodiment.

FIG. 30A through 34 illustrate a delivery method to retract, displace, or compress prostatic tissue according to an embodiment.

### DETAILED DESCRIPTION

The following description of the preferred embodiments of the invention is not intended to limit the invention to these preferred embodiments, but rather to enable a person of ordinary skill in the art to make and use this invention. Disclosed herein are systems and methods for treating conditions wherein a tissue (e.g., the prostate gland) has a) become enlarged and/or b) undergone a change in form, position, structure, rigidity or force exertion with respect to another anatomical structure and/or c) has begun to impinge upon or compress an adjacent anatomical structure (e.g., the urethra).

Anchors disclosed herein can be placed using steps of a method for compressing an anatomical region using a delivery device according to the following general description. Anchors of certain embodiments can be placed using variations of this general description. The delivery device is introduced in an anatomical region such that distal end of the delivery device is located adjacent to a target anatomical region to be treated. In one embodiment of a method, the delivery device is introduced transurethrally into the prostatic urethra. Thereafter, a puncturing element is advanced to puncture the anatomical region. In this example, the puncturing element punctures the prostate gland such that the distal end of the puncturing element is located in the pelvic cavity. The delivery device can include an anchor of certain embodiments herein, the anchor being carried by the puncturing element. The puncturing element can include a pusher element that can facilitate delivery and placement of certain anchors. Other elements that reversibly lock certain parts of the delivery device relative to each other can also facilitate delivery of anchors. An imaging modality can be used to verify the accurate placement and working of the delivery device and the anchor. In certain embodiments, the pusher element is pushed in the distal direction to push at least part of an anchor out of the puncturing element. In certain cases, at least part of the anchor is thereby deployed in the anatomy (e.g. in the pelvic cavity surrounding the prostate gland). Thereafter, the puncturing element is withdrawn by pulling it in the proximal direction. In embodiments in which part of the anchor includes a tensioning member, the delivery device can include a tension element that is used to set tension on such a tensioning member. For example, a tensioning element can be pulled in the proximal direction to set the tension. In certain embodiments, the tension element is pulled further in the proximal direction to compress the anatomical region between proximal and distal parts of an anchor. In certain embodiments, the proximal part of the anchor is then securely locked onto the tension member. Then, the proximal part of the anchor may be detached from the delivery device in certain embodiments. The detachment can be performed by a variety of mechanisms including, but not limited to, the anchor detachment mechanisms disclosed elsewhere in this patent application. In some embodiments, any excess length of the tension member is removed. This removal can be done using a variety of methods including, but not limited to, the methods disclosed elsewhere in this patent application such as cutting, delinking, melting, and breaking Thereafter, the delivery device is withdrawn from the anatomy. It should be understood that these deployment steps may be repeated in the same, opposing, or neighboring tissues to essentially tack up the encroaching tissue (e.g. prostatic tissue, tumor, relaxed tissue, expanded tissue or growth). It may be desired that over time the anchors become completely embedded within the tissue and covered to prevent encrustation, clotting, or other tissue or body-fluid interaction—this may be facilitated by the processes, therapeutic agents and coatings described elsewhere in the application. Although these anchors are shown on either side of the tissue, it may be possible to deploy either or both of them within the body of the tissue itself to help bury them and eliminate the possibility that they may interact with other parts of the body. It should further be noted that in the case of application to the prostate, that this technique may be used on any of the lateral or middle lobes to compress or hold the prostate gland away from the lumen of the urethra.

As shown in FIG. 1 A-G, in some embodiments of the invention, helical or coiled anchors can be employed. Such anchors can be deployed by a variety of delivery mechanisms, including rotating delivery mechanisms, and straight, hollow delivery mechanisms such as a needle or trocar in which a helical structure 100 can be stored in a linear configuration. The helical structure 100 can penetrate a prostate partially (FIG. 1A) or (FIG. 1B) through a target structure or tissue plane such as the capsule of a prostate, and can also be deployed entirely outside a target structure or tissue plane such as outside of the prostate (FIG. 1C), distal from the delivery trajectory. FIG. 1 A-C depict a lobe of the prostate gland PG as the target tissue. In FIG. 1B, the helical structure 100 is depicted as anchored to the prostatic capsule. The helical structure provides the ability to engagably secure to a tissue plane which location is not precisely known. By compressing such helical or coiled anchors, tension on a connecting member, such as a suture, can be provided. For example, by pulling the connecting member 150 proximally, the helical structure 100 can be compressed against tissue such as the prostatic capsule PC or the prostatic gland PG. When the connecting member 150 is tethered to proximal anchor, the compression on the helical structure 100 creates tension in the connecting member between the helical structure 100 and the proximal anchor. Proximal anchors are described in more detail in other embodiments disclosed herein. FIG. 1D depicts an anchor in which the coiled structure 100 is generally in the same plane and thus most of the coil can be placed in contact with tissue, thereby increasing the footprint of this distal section of the anchor against tissue. In the embodiment shown in FIG. 1E and 1E′, the helical structure 100 is depicted as having its distal end anchored to bone and its proximal end as anchored to the prostatic capsule PC. In FIG. 1E, the helical structure 100 is stretched to extend between the bone and the prostatic capsule PC. In FIG. 1E′, the spring-like structure 100 has been released and allowed to retract, which pulls the prostatic capsule PC towards the bone and help open the urethra.

Another embodiment of a coiled anchor in an alternative orientation is shown in FIG. 1F through 1F″′. In this embodiment, the distal end 100 of the anchor begins to coil up as it is pushed beyond the end of the delivery mechanism 200 and into the anatomy beyond the prostatic capsule as depicted in FIG. 1F and FIG. 1F′. As more of the anchor is pushed beyond the end of the delivery mechanism 200, the proximal end 190 of the anchor can emerge from a slot 210 along a portion of the length of the distal section of the delivery mechanism 200 as depicted in FIG. 1F″. The proximal end 190 of the anchor can include a preformed leg or tail that is capable of being held in a straightened configuration until it emerges from the delivery mechanism 200, at which point it assumes a comparatively curved configuration as depicted in FIG. 1F″′. The anchor provides a custom-fit or one-size-fits-all approach in that the amount of curved section versus the straight section can vary based on the tissue thickness between the urethra and capsule of the prostate. The distal end 100 of the anchor can include barbs or other structure that prevent the coil from unwinding and thereby allow the anchor to maintain tension on tissue. Further, pushing on the proximal tail 190 of the anchor can force more of the anchor beyond the prostatic capsule, which would reduce the length of the anchor in tissue. That is, the proximal tail 190 and the distal coil 100 would be closer together and the barbs or other structure that prevent the coil from unwinding would keep the tissue in compression. This and other embodiments disclosed herein provide a means of biasing the distal end of the anchor to compress, displace, or otherwise modify tissue. The displacement of the urethra can be adjusted over time by displacement of the proximal tail toward the distal coil (e.g., inflation of a balloon in the urethra).

FIG. 1G, 1G′ and 1G″ show an embodiment of an anchor that can be used to put tension on a connecting member 150 by a rotational motion. Similar to other embodiments, there is a proximal tail 190 and a distal coil 100. In this case, the distal coil 100 has a corkscrew-type configuration that allows the depth of the coil to be set in the tissue by rotating the anchor such that the distal coil “screws” through the prostatic capsule or other tissue plane. A non-circular cross section for at least the proximal tail 190 can facilitate the rotational motion. Any of the implants can be resorbable or biodegrable with a degradation profile that allows the implant to dissolve after the compressed tissue of the prostate gland has atrophied.

As shown in FIGS. 2 A-B′, in some embodiments of the invention, multiple anchors can be provided pre-assembled on a connecting member in series within a puncturing element, wherein the distance between the anchors is adjustable, for instance by at least one of the anchors being slideably included on the connecting member for successive deployments. A mass of tissue can be captured between two of the anchors 400 and 410 by delivering one anchor to a position distal of the mass of tissue, and the other anchor to a position proximal to the mass of tissue. As one embodiment, the anchors 400 and 410 can be stainless steel, stamped from sheet. As one embodiment, the connector 150 can be injection molded polyester. A compressive force can be exerted on the mass of tissue by moving at least one of the anchors over the connecting member in the direction of the other anchor.

To maintain the compressive force, the moving anchor or anchors can be locked onto the sliding member by a variety of mechanisms, as illustrated in FIG. 2A-B. FIG. 2A illustrates an embodiment of a push-nut or zip-tie like locking mechanism. The anchor system includes a proximal anchor 400 and a distal anchor 410. Distal anchor 410 includes a lock mechanism 415 that in FIG. 2A is depicted as a cut-out flap that allows connecting member 150 to travel only one way through the distal anchor 410, analogous to a push-nut or zip-tie. FIG. 2A depicts the connecting member 150 as having a cap 155 against which the proximal anchor 400 can seat, but other mechanisms for fastening the proximal anchor 400 to the connecting member 150 are contemplated as described herein.

FIG. 2B illustrates an embodiment of a circumferential pivot-to-clamp mechanism. Clamping anchor 300 resides on the connecting member 150 and clamps onto the connecting member 150 when anchor wings 310 are pivoted from one configuration to another. It can be preferable that the unclamped configuration is such that the anchor wings 310 are substantially parallel to the connecting member 150 and the clamped configuration is such that the anchor wings 310 are substantially perpendicular to the connecting member 150. Thus, when the clamping anchor 300 is clamped, the perpendicular anchor wings 310 provide a larger footprint to present to the tissue being displaced or compressed. The pivoting action engages the central section of the clamping anchor 300 with the connecting member such that the clamping anchor 300 is secured in place. Otherwise, the clamping anchor 300 can be free to slide along the connecting member 150.

These and other embodiments employing slide and lock anchors can advantageously be used to load multiple anchors on a single connecting member, reduce delivery sheath size, simplify delivery mechanism and obtain automatic locking upon deployment of an anchor.

As shown in FIG. 3 A-C, in some embodiments, a profile of a shaft of a device can be reduced by employing an anchoring system having a primary anchor 500 and a secondary anchor 590 that can be attached to each other by a length of suture 550 (FIG. 3A). The primary anchor 500 can be pre-bonded to the suture 550 and can be slid over a needle or trocar 560. The primary anchor 590 can be pushed off the needle or trocar 560 at the time of delivery by a pusher 530 (FIG. 3A and 3C). The primary anchor 500 can have a tail 505 that can be pre-formed to take a curved position if unconstrained (FIG. 3B), but can get straightened out when assembled onto the needle or trocar 560. This can provide a feature that facilitates flipping and engagement of the anchor inside of tissue or on the other side of a tissue plane once the anchor is pushed off the trocar.

FIG. 4 A-B depict a variation of the embodiments of FIG. 3A-3C in which the primary anchor 500 can be prebonded to the suture 550 and can be located inside a needle or trocar 560. The secondary anchor 590 can be housed outside the needle or trocar 560 and can be supported by a pusher 530 (FIG. 4A). The secondary anchor 590 can completely surround the suture 550 after assembly (FIG. 4B), which can reduce the risk of detachment from the suture 550.

As shown in FIG. 5A-5C, in some embodiments a tissue anchor 600 can be employed having a rim 610 or depth stop 620 for the purpose of preventing undue protrusion of the anchor 600 into surrounding tissue, for instance in the case of providing an anchor to the capsule of a prostate, without creating a risk of penetrating adjacent rectal tissue. The anchor 600 can be connected to a proximal anchor via a connecting member 150.

As is shown in FIG. 6 A-B, in some embodiments a spring-loaded member 720 can provide tension to a connecting member 150 between two tissue anchors 700, 790. As shown in the embodiment in FIG. 6A, a pair of tissue anchors 700, 790 can be positioned on the same side of a tissue plane or separate tissue planes as desired to achieve the intended effect, and be connected by a connecting member 150. The anchor section, indicated by the arrows A and A′ in FIG. 6 can be looped behind a second tissue plane. A coiled tensioning member 720 can be positioned over the anchor section, and after expansion, increase the length of the anchor section, as shown in FIG. 6B, thereby pulling the two tissue planes in closer proximity.

As is shown in FIG. 7 A-B, in some embodiments of the invention a body lumen, such as a urethra, can be enlarged by the employment of a suture 150 only, in which the suture has been knotted 151, or by a combination of a suture 150 and an adhesive 160 placed at the point where the suture 150 is intended to secure tissue.

In the embodiment of FIG. 7A, a suture 150 can be introduced through the wall of the urethra UT, directed in a loop behind the prostatic capsule and redirected to the lumen of the urethra UT, where the suture ends are tied together under tension, putting a compressive force on the prostate tissue. In the embodiment of FIG. 7B, the suture 150 can be anchored to the wall of the urethra UT, directed to and through the prostatic capsule and fixated under tension by the application of a surgical glue 160.

As shown in FIG. 8A, in some embodiments a capsular tab 800 to be placed at the capsule side of the prostate can be attached to an elongated member 830, such as rod or wire using a sacrificial bond or weld 840. These embodiments can advantageously be employed to avoid the use of a needle to place the capsular tab, thereby avoiding the cost involved in the manufacture of such needles. The elongated member 830 can be formed to have a pre-set curvature and be housed in a lumen when not in use. The elongated member 830 can be made of any suitable, preferably biocompatible material, including polymeric and metallic substances and compositions. In some embodiments, the elongated member 830 can be advantageously made of metallic alloys, such as Nitinol. The elongated member 830 can have a sharpened tip 835 to more effectively penetrate tissue. On deployment the elongated member 830 and capsular tab 800 can be driven out into the prostate and past the capsule. To deliver the capsular tab, the sacrificial bond or weld 810 can be broken such as by electrolysis. In an alternative embodiment, the elongated member can have a ring holding a section of the capsular tab. The ring can drive the capsular tab and the elongated member out onto the capsule of the prostate together. Upon retraction of the elongated member, the ring can allow the tail-end of the capsular tab to release, leaving the capsular tab on the capsule side.

As shown in FIG. 8B, in alternative embodiments, a capsular anchor 800 can serve as a leading point having a tip 805 that penetrates tissue, replacing the beveled portion of a delivery needle. The capsular anchor 800 can be connected to a hypotube 860. In some embodiments, the hypotube can be advantageously made of metals, such as stainless steel, or of metallic alloys, such as Nitinol. In some embodiments the tip 805 can be made of polymeric materials, such as polyethylene terephtalate, and can be shaped or overmolded onto the capsular anchor. Upon deployment, the hypotube 860 can drive the capsular anchor 800 out into tissue and past the capsule. Upon retraction of the hypotube 860, the capsular anchor 800 secures tissue. In yet another embodiment the tip can be bioabsorbable.

In an embodiment of a method to reduce prostatic obstruction, tissue can be pulled away from the urethra via an extra-prostatic approach. A needle or trocar can be inserted transdermally through the obturator foramen or cranial to the superior pubic ramus and into the prostate. A tethered tissue anchor can be deployed from the tip of the needle or trocar into the prostate. The needle can be partially withdrawn, and the tether drawn taught to seat the prostatic anchor against the interior surface of the capsule. A second tissue anchor can then be delivered into the peri-prostatic fascia or the pelvic fascia.

In another embodiment of method to reduce prostatic obstruction, a capsular anchor can be delivered through the prostatic capsule using a pressurized propulsion mechanism. The pressurized propulsion system can employ a piston assembly to propel the capsular anchor without discharging the propulsion medium, like a compressed gas, into the body. After firing, the trajectory of the capsular anchor can be limited by a tether, such as a length of suture. In another embodiment a pressurized propulsion mechanism can be used to propel a needle or trocar housing the capsular anchor.

In some embodiments, as illustrated in FIG. 9 A-D, a capsular anchor on a device to reduce prostatic obstruction can be constructed of a single, deformable member 900. The member can be used to penetrate the prostatic capsule PC, and subsequently deformed on the outside of the capsule to form an anchor. FIG. 9A depicts an anchor in which the proximal part has a series of narrower segments 905, which provide a position at which the proximal part will more easily bend. Thus, the length of the member within tissue can be customized by bending the proximal part of the member 900 at the appropriate narrower segment 905. The distal section 910 of member 900 includes a wing 912 that allows the member 900 to penetrate the prostatic capsule PC but not be able to retract back through the prostatic capsule PC.

FIG. 9B depicts an expansion mechanism 930 on the distal section of the anchor 900 that is configured to deform and expand, increasing the effective footprint of the anchor on the prostatic capsule PC. In this embodiment, the proximal section of the anchor 900 includes a tail 920 configured to assume a curved configuration when released from a delivery device.

FIG. 9C and 9C′ depict an anchor in which the distal coil 915 is formed by physically deforming distal segments of the anchor against a forming feature (e.g., an anvil) located at the distal end of the delivery member 990. In this embodiment, the anchor 900 does not have a preformed coil at its distal tip, but instead the distal coil 915 is custom-formed by the features at the end of the delivery member 990. FIG. 9C′ depicts the anchor 900 in place in tissue, and include a proximal tail 920 that is also formed by the delivery member 990. FIG. 9D shows more detail of an embodiment of a helical or coiled anchor distal end 915 that can be formed in-situ by advancing a plastically deformable material against an anvil feature in a delivery member 990. The amount of coiled material can be adjusted to facilitate treatment of different thickness prostates.

In some embodiments an elastic deformation of a material can be used to exert a force that reduces prostatic obstruction.

FIG. 10A-C show embodiments of elastically deformed devices that can introduced into tissue under tension, and that after release from a delivery device can revert to an unconstrained configuration, thereby enlarging a constricted urethral channel. For example, FIG. 10A depicts a wire-like or ribbon-like device 1000 implanted in each of two lobes of the prostate gland PG near the urethra UT. As the device 1000 is allowed to assume its unconstrained configuration, it expands the urethral lumen as depicted in FIG. 10A′. FIG. 10B depicts a similar wire-like or ribbon-like device 1000 implanted at the urethral bend near the bladder. Straightening this bend can have the effect of relieving BPH symptoms. FIG. 10C depicts another placement of wire-like or ribbon-like devices 1000.

In some embodiments, anchoring of a tissue constraining member that relieves prostatic obstruction can be accomplished by barbs on the constraining member. FIG. 11A and 11B illustrate embodiments of barbed tissue constraining members. In FIG. 11A, retraction of a delivery member 1150 with respect to an anchor 1100 can deploy barbs 1155. In some embodiments natural movement of the prostatic tissue can promote advancement of the barbed sections of the constraining member into the tissue. In FIG. 11B, barbs 1155 on either end of an anchor 1120 allow the anchor to compress tissue. The depth of penetration of the barbed ends through tissue sets the amount of compression on the tissue.

In some embodiments, plastically deformable tissue constraining members can be formed to function as tissue staples. FIG. 12 A-C illustrate embodiments of such tissue constraining members 1200. The figures illustrate various placements of staple-like members 1200 that provide for expansion of the urethral lumen UT.

FIG. 12D illustrates an embodiment of an adjustable die 1250, suitable to form variable curvature staples 1200. Such customized staples can help reduce or minimize damage to tissue by matching the restraining force of the staple to the specific tissue geometry being restrained.

As shown in FIGS. 13A-E, in some embodiments, multiple capsular anchors can be provided in a device, for instance like a needle or trocar, loaded in series within the device for successive deployments.

FIG. 13A and 13A′ illustrate an embodiment of a device 405 capable of deploying the multiple anchors 400, 410 depicted in FIG. 2A. A wire 407 routes through each anchors hole 412 such that the wire can be used with each deployment. The wire can be a threaded member 490 which engages the distal anchor tab 410 via hole 412 and allows for the anchor to be positioned at the desired place in tissue via the stiffness imparted by the threaded member that allows the anchor to be pushed through tissue and/or held in place while a needle containing the anchor is retracted. The wire 407 is then pulled in tension while an anchor 400 is pushed in place to cinch the two anchors together. The proximal anchor 400 emerges from the device and can be pushed against tissue to compress or displace the tissue. The pushing also forced connector 150 through the one-way lock 415 of distal tab 410. When the two anchors are in the appropriate position the elongate member 490 is unscrewed, retracted back to the next anchor within the device and the next pair of anchors in the device can be deployed by the same mechanism.

FIG. 13B and 13B′ show an embodiment of a device in which multiple capsular anchors 1300 can be loaded in the delivery needle or trocar 1350 and released manually.

FIG. 13C shows an embodiment of a device having a slotted delivery needle or trocar 1459 into which multiple anchors 1400 can be loaded.

FIG. 13D shows an embodiment of a device having a delivery needle or trocar 1550 which can be preloaded with multiple implants 1500 advanced by an elongated member 1530 like a wire. Implants 1500 include a distal tab 1510 and a coil 1590, both of which features are disclosed in detail elsewhere herein.

FIG. 13E shows an embodiment of a device that can have a series of metallic implants 1600, like nitinol implants, in a needle 1630 having a slot 1610. The implants 1600 function to grip a suture 1650. The series of implants can be advanced out of the end of needle 1630 and deployed to compress tissue. Each implant 1600 can act as a suture lock such that tissue is compressed between suture segments locked by each implant.

As shown in FIGS. 14A-B, in some embodiments a handle on a device or a body of a device can contain multiple anchors.

FIG. 14A shows an embodiment of a device having a holder 1700 with multiple anchors 1710 that can be advanced by a spring-driven mechanism 1730.

FIG. 14B shows an embodiment of a device with a rotating holder 1800 that can contain multiple anchors 1810.

As shown in FIG. 15A-B, some embodiments can avoid the need for a hollow needle.

FIG. 15A shows an embodiment of an implants having sharp tips. The implants can register on a guidewire, which can drive the implants through tissue. A fixed length of a connecting member, such as a suture, can connect the implants. Looking from anterior to posterior, capsular anchors with a fixed connecting member therebetween can be pushed simultaneously with penetrating members from the urethra through the right and left lateral lobes of the prostate to hold back the encroaching tissue.

FIG. 15B shows an embodiment of an implant 2000 located on the exterior of a delivery device, such as a sharpened rod or a trocar 2050. Implant 2000 is connected to a suture 2010, which in turn can be connected to other implants. Suture 2010, like the implant, is outside the trocar 2050.

As shown in FIG. 16-18, in some embodiments a suture loop or double suture configuration can be directed through the prostatic tissue, from the extra-capsular space to the urethral lumen or vice versa. A compressive force can be exerted on the tissue by placing tension on the sutures. Alternatively, these elements can be combined with the adjustable coiled anchors described elsewhere in the application to provide a one-size-fits-all configuration.

FIG. 16 shows an embodiment of a suture loop 2150 that can be tightened by two spring-loaded mechanisms 2100 like those disclosed elsewhere herein, such as Nitinol coils, outside the prostatic capsule PC.

FIG. 17 shows an embodiment of a suture loop that can be fitted with T-bars 2200, pierced outside the prostatic capsule PC and tightened from the center.

FIG. 18 shows an alternative embodiment of a suture loop that can be anchored with extra-capsular anchors 2300.

As shown in FIG. 19-21, in some embodiments an anchor or locking device can be positioned on a suture, a suture loop or alternative suture configuration directed through a prostatic urethra.

FIG. 19 shows an embodiment of a suture configuration that can be anchored by two extra-capsular anchors 2400 where a suture configuration can be tightened by a sliding tightener 2420.

FIG. 20 shows an embodiment of configuration wherein two sutures A, B can each be anchored to a different extra-capsular anchor 2500, 2500′, and where tension on the sutures can be provided by a sliding suture through the tightener 2550 or sliding the tightener along the suture. The sliding tightener 2550 is crimpable or otherwise securable to suture B when the suture has been sufficiently tightened.

FIG. 21A and 21B show an embodiment of a tightener 2420 in which a tortuous path can provide a sliding/locking mechanism to tighten a suture configuration that can be anchored to two extra-capsular anchors 2400. FIG. 21A shows the tightener 2420 in a pre-crimped position prior to the path becoming tortuous. After the diameter of the tighteneer 2420 is decreased, the suture path through the tightener will become more tortuous. FIG. 21B shows a tightener 2420 that becomes more tortuous as it is “crushed.”

FIG. 22A, 22A′ and 22A″ show embodiments of a suture loop that can be anchored by two extra-capsular anchors 2500, 2500′, wherein at least one of the anchors can be located on the suture 2550 in a sliding manner, and where the suture loop can be tightened by advancing the suture through at least one of the anchors. This embodiment can optionally include a suture lock 2555. In FIG. 22C, at least one of the sliding anchors can be configured analogous to a clothespin. FIG. 22D depicts placement of two anchors 2500, 2500′ using a device including two delivery needles 2530 and 2540. Each of the needles 2530 and 2540 can include a slot, which allows suture 2550 to exit the needle after deployment.

FIG. 23 shows an embodiment in which the delivery device 2690 includes hypodermic tubing 2630, which houses a capsular anchor assembly 2600 and a guidewire component 2610. FIG. 24 A-C show different cross-sectional views to illustrate how the guidewire 2610 can be housed internally in the delivery device along with the capsular anchor assembly 2600. Preferably, the guide wire 2610 has a round cross-section and is formed from a stainless steel material or a comparatively hard plastic material.

The delivery sequence for the embodiments illustrated in FIG. 23 and FIG. 24A-C can be as follows: (1) the delivery device 2690 is positioned at the target location on a prostatic lobe; (2) the guidewire 2610 is delivered through prostatic tissue and past the capsular surface; (3) the hypodermic tubing 2630 is delivered over the guide wire 2610 until it has penetrated past the capsular surface of the prostate; and (4) the hypodermic tubing 2630 is pulled back into the delivery device 2690 to deliver and seat the capsular tab 2600 against the capsular surface or within the prostate.

Regarding the individual delivery steps, the first step has certain challenges that can be addressed as described herein. One of the challenges of delivering the guidewire component is to maintain the position of the delivery device relative to the prostatic lobe while the guidewire is being advanced. This can be done by maintaining compression on the lobe and manually feeding the guidewire through the prostate. A guidewire with a sharp end and small diameter can penetrate through the prostate with comparatively less force than is required to compress the lobe. This force differential will maintain the position of the delivery device relative to the prostatic lobe. Alternatively, a sharp penetrator on the shaft or at the leading edge of the hypodermic tubing could make pilot hole in a position, perpendicular to the delivery device, for the guidewire to enter the prostate. This can ensure that the trajectory of the guidewire is correct. Further, features incorporated on the delivery device can help anchor the device to the prostatic lobe. Such features could be a clamp mechanism which when pushed against the prostatic lobe clamps onto the tissue, as depicted in FIG. 25A and 25B. Such a clamp mechanism could be part of the tip of the device or a separate clamping feature attached to the tip of the device. Still further, a vacuum system could be used to maintain the position of the delivery device with respect to the prostatic lobe.

Regarding the second step, the guidewire component can be delivered through the prostate by manually feeding the wire through the prostate (through the device out the back of the handheld portion of the delivery device) or using a spring-loaded mechanism to deliver the guidewire. Once delivered, secure attachment to the capsular surface may be required to assist with the next step. Mechanical features preventing the guidewire from pulling back the prostate can help with such a secure attachment. Alternately, a guidewire to having shape memory features creating a geometry for secure attachment surface can be used. FIG. 26 illustrates these embodiments.

In the third step the hypodermic tubing is manually fed along the guidewire until it has penetrated past the capsular surface. As the hypodermic tubing is advanced, the guidewire feeds back into it. The pullback features in the guidewire would be pulled into the hypodermic tubing as it is advanced forward. In the fourth step, the hypodermic tubing is manually pulled back to unsheath the capsular tab. Alternately, the hypodermic tubing could use a spring-loaded mechanism (either pre-loaded or loaded as part of the delivery of the hypodermic tubing in step 3) to deploy the capsular tab.

FIG. 27 through 34 depict another embodiment of the invention in which anchor parts are delivered through two different penetrating members. FIG. 27A and 27B illustrate two views of a stamped penetrating member assembly 2800. A channel 2810 is formed by stamping sheet metal such as stainless steel. A wire 2830 is captured by stamped features 2840. The majority of the penetrating member length has a ribbon-like cross-section. The profile, or cross-section, of the end of the penetrating member can have a concave or double concave configuration. The tri-facet tip 2805 is oriented to drive through tissue.

FIG. 28 illustrates a distal anchor portion 2900. The distal anchor portion 2900 can be formed from tubing such as stainless steel tubing. The tubing can be crimped onto a segment of suture 150 and includes a hole 2930 for the suture 150 to exit. The tubing can include a feature 2920 to facilitate crimping, such as a projection into the tubing or a hole for suture to engage when the suture is crimped. The anchor further includes an angled end 2990 that facilitates ejection of the anchor from the penetrating member. FIG. 29A and 29B illustrate a proximal anchor section 3000, which includes a raised pulley feature 3010, holes for the suture 3020, a lock feature for one-way cinching 3030, and optionally an arm 3035 to stiffen the lock feature. The tip 3038 of the lock feature can be bent down to increase engagement with suture, as depicted in FIG. 29A. The features on the proximal anchor enable one-way cinching of suture. Further, the tip can have features at its end that improve the engagement with suture. The suture can be pulled in one and when tension pulls the suture in the opposite direction, the leading edge of the spring, and any feature thereon, stops the suture from progressing backwards. The edge is formed such that it presses down on the suture.

FIG. 30A and 30B illustrate, respectively, a side view and an angled view of the distal end of the first penetrating member 2800. The penetrating member 2800 holds the first anchor portion 2900 housed in the channel 2839 of the penetrating member. The penetrating member 2800 deploys through the prostate.

FIG. 31A and 31B illustrate, respectively, a side view and an angled view of the distal end of the first penetrating member 2800 from which a distal anchor portion 2900 and a suture 150 have been ejected. The distal anchor portion 2900 is ejected by advancing the wire 2830. The angle on the tip of the wire 2830 hits the angled end of the anchor and ejects the anchor.

FIG. 32A and 32B illustrate, respectively, a side view and an angled view of the distal end of the second penetrating member 2800′. The penetrating member 2800′ holds the proximal anchor 3000 housed in the channel 2810′. The penetrating member 2800′ deploys through the prostate.

FIG. 33A and 33B illustrate, respectively, a side view and an angled view of the distal end of the second penetrating member 2800′. The proximal anchor 3000 is ejected by advancing the wire 2830′. The angle on the tip of the wire hits the angled end of the anchor, and ejects the anchor.

FIG. 34 depicts distal anchor 2900 and proximal anchor 3000 in use, compressing tissue. The delivery tool can have an adjustable length between the penetrating members and deploy two anchors at once. In some instances, it may be desirable to measure the prostate thickness and urethra length prior to setting the distance between the penetrating members.

Certain embodiments relate to a system for treatment of a prostate. The system includes a first anchor, a second anchor, and a tensioner. The first anchor, second anchor, and tensioner are connected to a suture. The system also includes a delivery device having a needle configured to place the first anchor and second anchor outside the prostatic capsule from a position within the prostatic urethra. The tensioner is configured to increase the tension on a length of the suture positioned between the first and second anchor after each anchor has been placed outside the prostatic capsule.

Certain embodiments relate to a system for treatment of a prostate. The system includes a first anchor connected via a length of a first suture to a tensioner and a second anchor connected to a second suture. The second suture is slidably engaged to the tensioner in a first tensioner configuration and fixedly engaged to the tensioner in a second tensioner configuration. The first anchor and second anchor are each placed outside the prostatic capsule and the tensioner is placed near a urethral surface of the prostate.

Certain embodiments relate to a method of placing the anchors described herein by penetrating the prostatic capsule with a penetrating member. The penetrating member is a needle, a wire, a trocar, or any of the anchors.

Certain embodiments relate to a system for treatment of a prostate. The system includes a first anchor configured to be placed on an outer surface of a capsule of the prostate. The first anchor is fixed to an end portion of a flexible connector. A second anchor is configured to be placed on an outer surface of a capsule of the prostate, the second anchor slidably engaging the flexible connector. The second anchor is configured to allow the connector to slide only in one direction and wherein a portion of the connector is placed across a urethral surface of the prostate

The examples and illustrations included herein show, by way of illustration and not of limitation, specific embodiments in which the subject matter may be practiced. Other embodiments may be utilized and derived therefrom, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is in fact disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art.

What is claimed is:

1. A system for treatment of a prostate, comprising:
a first anchor configured to be placed on an outer surface of a capsule of the prostate, the first anchor fixed to an end portion of a flexible connector; and
a second anchor configured to be placed on an outer surface of a capsule of the prostate, the second anchor slidably engaging the flexible connector;
wherein the second anchor is configured to allow the connector to slide only in one direction and wherein a portion of the connector is placed across a urethral surface of the prostate.

2. The system of claim 1 wherein the first anchor is configured to toggle about the connection point with the end portion of a flexible connector.

3. The system of claim 1 wherein the second anchor is configured to create a 180 degree bend in the flexible connector.

4. The system of claim 1 wherein the second anchor comprises a rounded projection and a stopping latch.

5. The system of claim 4 further comprising a wire including an angled distal end, wherein the angled distal end of the wire cooperates with the stopping latch of the second anchor to eject the second anchor from the system.

6. The system of claim 1 wherein an end of the first anchor is angled.

7. The system of claim 6 further comprising a wire including an angled distal end, wherein the angled distal end of the wire cooperates with the angled end of the first anchor to eject the first anchor from the system.

8. The system of claim 1 wherein multiple second anchors are loaded within the system.

9. The system of claim 1 wherein the first anchor and the second anchor are carried by a penetrating member.

10. The system of claim 9 wherein the first anchor is ejected from a portion of the penetrating member proximal to a distal end portion of the penetrating member.

11. The system of claim 9 wherein the second anchor is ejected from a portion of the penetrating member proximal to a distal end portion of the penetrating member.

12. A method of placing anchors proximate prostate tissue, comprising:
placing a delivery system proximate a prostate gland, the delivery system comprising a penetrating member, a first anchor, a second anchor, and a flexible connector, wherein the first anchor is fixed to the connector and the second anchor is configured to allow the connector to slide only in one direction;
advancing the penetrating member to penetrate the prostatic capsule at a first location;
deploying the first anchor to a position proximate an outer surface of the prostatic capsule;
advancing the penetrating member to penetrate the prostatic capsule at a second location; and
deploying the first anchor to a position proximate an outer surface of the prostatic capsule, wherein the connector connects the first anchor and second anchor and is positioned such that a segment of the connector engages a portion of the prostatic urethra of the prostate gland.

13. The method of claim 12 wherein the system further comprises a deployment wire and the first anchor is deployed by engaging an end of the deployment wire with an end of the first anchor.

14. The method of claim 12 wherein the system further comprises a deployment wire and the second anchor is deployed by engaging an end of the deployment wire with an end of the second anchor.

15. The method of claim 12 further comprising setting a tension on the connector.

16. The method of claim 15 wherein the tension is maintained by features on the second anchor.

17. The method of claim 16 wherein tension is maintained by the cooperation of a rounded projection and a stopping latch on the second anchor.
